(19) United States
(12) Patent Application Publication
(10) Pub. No.: US 2017/0345274 A1
(43) Pub. Date: Nov. 30, 2017

(54) NECK POSTURE RECORDING AND WARNING DEVICE

(71) Applicant: General Scientific Corporation, Ann Arbor, MI (US)

(72) Inventor: Byung J. Chang, Ann Arbor, MI (US)

(21) Appl. No.: 15/167,018

(22) Filed: May 27, 2016

(51) Int. Cl. G08B 21/04; G08B 3/00; G08B 5/36; A61B 5/11; A61B 5/00

(52) U.S. Cl. CPC G08B 21/0446; A61B 5/1121; A61B 5/6803; G08B 5/36; G08B 3/00; A61B 5/1116; A61B 5/742; A61B 5/7405; A61B 2560/0475; A61B 2562/0219; A61B 2560/0214

(57) ABSTRACT

A miniature electronic device senses head tilt and informs the user so as to avoid neck strain. The device may inform the user via various audio/visual alarms, and my sound based upon angle, duration, or both. Wireless interfaces to other devices may be provided. The device includes a housing adapted for mounting on a wearer's head so as to move in a manner corresponding to front-to-back head movements, an input for receiving information corresponding to a particular forward head tilt angle, and a sensor that detects when a wearer of the device tilt their head at an angle equal to or greater than the particular forward head tilt angle. In particular embodiments the forward head tilt angle is in the range of 5 and 35 degrees, and more particularly approximately or equal to 20 degrees. The sensor may include a gyroscopic sensor, an acceleration sensor or, more preferably, a combination thereof.

### FIELD OF THE INVENTION

This invention relates generally to ergonomics and, in particular, to apparatus and methods to discourage prolonged head tilt to minimize and avoid neck and back strain.

### BACKGROUND OF THE INVENTION

Many dentists and surgeons use head-mounted loupes to enhance vision during delicate procedures. To maintain depth perception, loupes are binocular, and are usually attached to eyeglass lenses or frames. Loupe varieties include front-lens-mounted (FLM) types which may flip up and down, and through-the-lens (TTL) types that are inset within the lens of the glasses. Loupes are often custom made for the individual practitioner, taking into account their corrected vision, interpupillary distance and desired focal distance. Multiple magnification powers are available in all types.

If eyeglass-mounted loupes are not designed correctly, the wearer bends his/her neck too often, and/or at too great an angle, leading to neck and back strain. One important consideration is declination angle, which may be defined as the angle between a reference line that connects the top of the ears (where the frame arm rests) to the corner of the eyes, and the optical axis of loupe oculars (FIG. 1). Frame temple arms may be used as the reference line if the temple arms are mounted at the same level with eyes, keeping in mind that the temple arms of some frames are higher than eye level. Note that the reference line angle is unique to each person. The posture angle will be smaller, as the head tilt angle increases. For the person in FIG. 2, the posture angle is 23 degrees, the reference line angle 12 degrees and the head tilt angle is 20 degrees. Thus, the minimum rotation angle of his eyes to avoid neck strain is 35 degrees.

Ergonomic posture is not a natural posture. Our natural instinct is to tilt our heads and twist our bodies to get a closer view to our work. Sitting upright and rotating the eyes down rather than tilting the head takes practice. Unfortunately, many clinicians are still using traditional TTL and FLM loupes with relatively small declination angles on the order of 20-25 degrees, which force users to tilt their heads excessively. Since traditional loupes support natural behavior, it is easier to use traditional loupes (FLM or TTL) with small declination angles, at least at first. Pain in the neck, shoulders and upper back due to the use of non-ergonomic loupes with small declination angles may not develop for several years. Since young clinicians (dental students and surgical residents) do not feel this pain initially, they may start their careers using traditional, non-ergonomic TTL loupes. However, it has been reported that excessive head tilt can develop into chronic pain and injury.

According to one recommended model, the maximum sustainable head tilt should be less than 20 degrees (See, Valachi B, Practice dentistry pain-free, Posturedontics Press, Portland, Oreg., 2008: 26, 69). Valachi found that loupes profoundly influence the musculoskeletal health of clinicians. She found a majority of loupes on the market are not ergonomic and place the user in an unsafe forward head-tilt posture (greater than 20 degrees). In international consultations, Valachi consistently found that clinicians with chronic neck, shoulder and upper back pain have used non-ergonomic loupes with small declination angles. By switching to properly designed and adjusted ergonomic loupes, however, they were able to eliminate their chronic neck, shoulder, and upper back pain.

Devices have been described to monitor and maintain proper posture. Published U.S. Application 2015/0374266 describes a device for detecting an unacceptable deviation from a neutral spine position. The device comprises a first sensor configured to determine the change in orientation, a second sensor configured to determine the change in curvature, a signaling mechanism configured to issue an alert to the user, an attachment element configured to maintain the device on the user, and a controller configured to determine that an unacceptable deviation has occurred based on readings of the first and second sensors. However, the device is configured to determine changes in orientation and curvature of the user's back, not neck bending or head tilt.

U.S. Pat. No. 5,425,378, entitled “Advanced posture-monitoring device” discloses a head-worn device that detects head tilt. However, this apparatus is directed to appearance, and the tilt limit is selectable by the user in a working range which includes positive and negative values from 2 degrees to 7 degrees of tilt.

Tilt sensors have been attached to eyeglass frames, but again they are for purposes other than ergonomics. For example, U.S. Pat. No. 8,337,014 teaches an electronic eyeglass frame with a tilt switch, but the switch is for controlling an electro-active lens. By having a tilt switch of an electro-active lens operate in this manner, a person can control his lens without having to manually touch a switch on the frame. WO 2012018409 resides in a tilt compensation system that provides an inexpensive and effective solution to remove stereoscopy artifacts, particularly artifacts associated with head tilt issues. WO 2013140744 is directed to a head-mounted display is provided which includes a detector for detecting an angle of tilt of the main body of the head-mounted display with respect to the horizontal plane xy, and an output unit comprising LEDs for outputting an indication of the angle of tilt.

### SUMMARY OF THE INVENTION

This invention resides in a device, mountable to eyeglass frames, that senses head tilt and informs the user if they may experience neck strain. The device may inform the user via various audio/visual alarms, and my sound based upon angle, duration, or both. Wireless interfaces to other devices may be provided.

A neck posture monitoring device according to the invention includes a housing adapted for mounting on a wearer's head so as to move in a manner corresponding to front-to-back head movements. The device includes an input for receiving information corresponding to a particular forward head tilt angle, and a sensor that detects when a wearer of the device tilt their head at an angle equal to or greater than the particular forward head tilt angle.

In particular embodiments the forward head tilt angle is in the range of 5 and 35 degrees, and more particularly approximately or equal to 20 degrees. The device may further include an alarm that is activated when the sensor detects when a wearer of the device tilts their head at an angle equal to or greater than the particular forward head tilt angle. The alarm may include a light source for generating a visible alarm or a transducer for generating an audible alarm. The audible transducer may form part of an earphone or earbud.

The housing for the device may be adapted for mounting on a wearer's head includes one or more fasteners for attachment to eyeglass frames, or may be adapted for mounting on a wearer's head via an on-ear or in-ear housing. A real-time clock may be provided for determining date and time, along with a memory for recording a wearer's head tilt angle as a function of time. An alarm may be activated when the wearer meets or exceeds the particular forward head tilt angle a specific number of times over a specific period of time, and/or when the wearer meets or exceeds the particular forward head tilt angle for a particular duration of time.

The device may further include an input for receiving information corresponding to a head neutral position, or an electronic level that operates in conjunction with the sensor to determine when a wearer of the device tilt their head at an angle equal to or greater than the particular forward head tilt angle. In the preferred embodiments the sensor is a gyroscopic sensor, and the device is configured as a self-contained battery operated device.

### BRIEF DESCRIPTION OF THE DRAWINGS

FIG. 1 is a diagram that illustrates declination angle and parameters other associated with head movement and tilt;

FIG. 2 is a drawing that shows a posture angle is 23 degrees, a reference line angle 12 degrees and a head tilt angle is 20 degrees, such that the minimum rotation angle of his eyes to avoid neck strain is 35 degrees;

FIG. 3 is a block diagram of important electronic components associated with the invention;

FIG. 4 a preferred embodiment of the invention mounted to eyeglass frames;

FIG. 5 is a drawing that shows an alternative embodiment of the invention in the form of an ear mounted device;

FIG. 6A is a diagram showing a user wearing an eyeglass-mounted sensor at a neutral posture;

FIG. 6B is a diagram showing a user wearing an eyeglass-mounted sensor at a maximum safe head tilt;

FIG. 6C is a diagram showing a user wearing an eyeglass-mounted sensor at an unsafe head tilt; and

FIG. 7 shows how an acceleration sensor and a gyroscopic sensor may be combined in accordance with the invention to achieve a more effective neck posture recording and warning device.

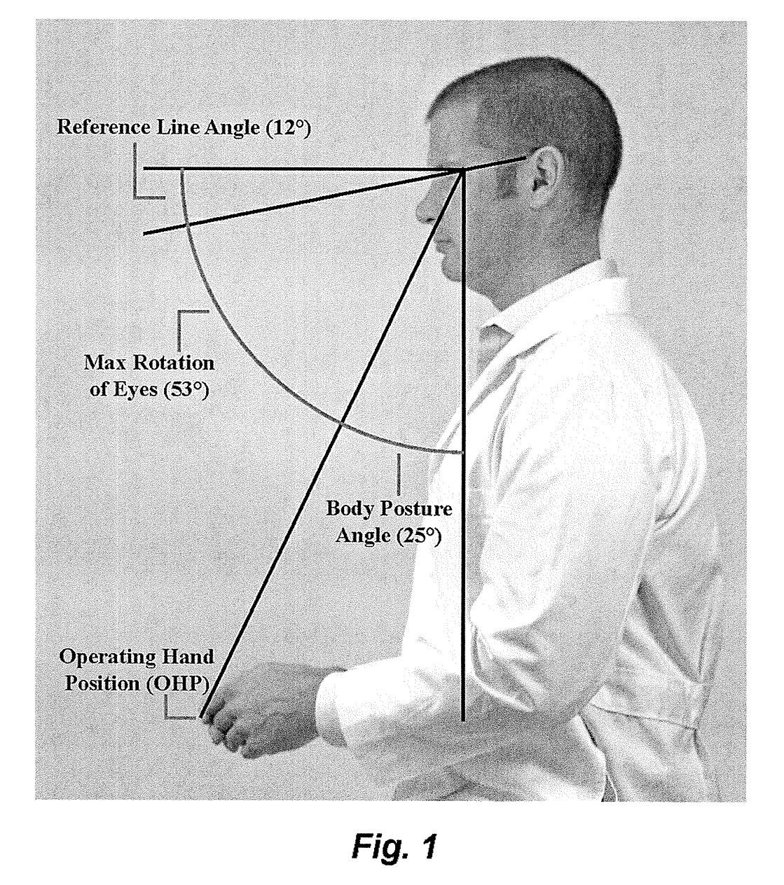

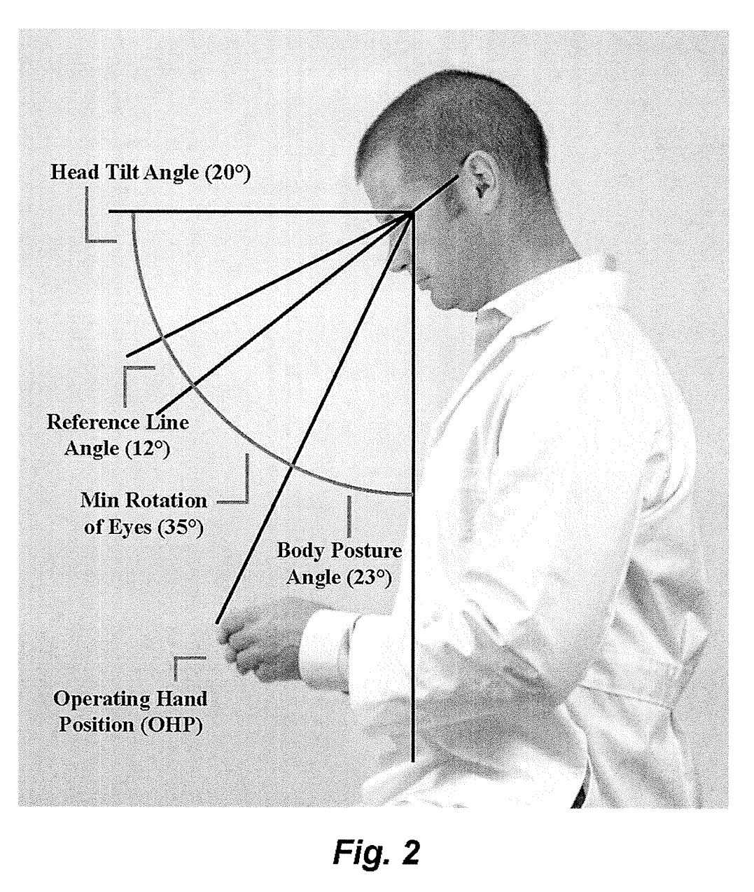

### DETAILED DESCRIPTION OF THE INVENTION

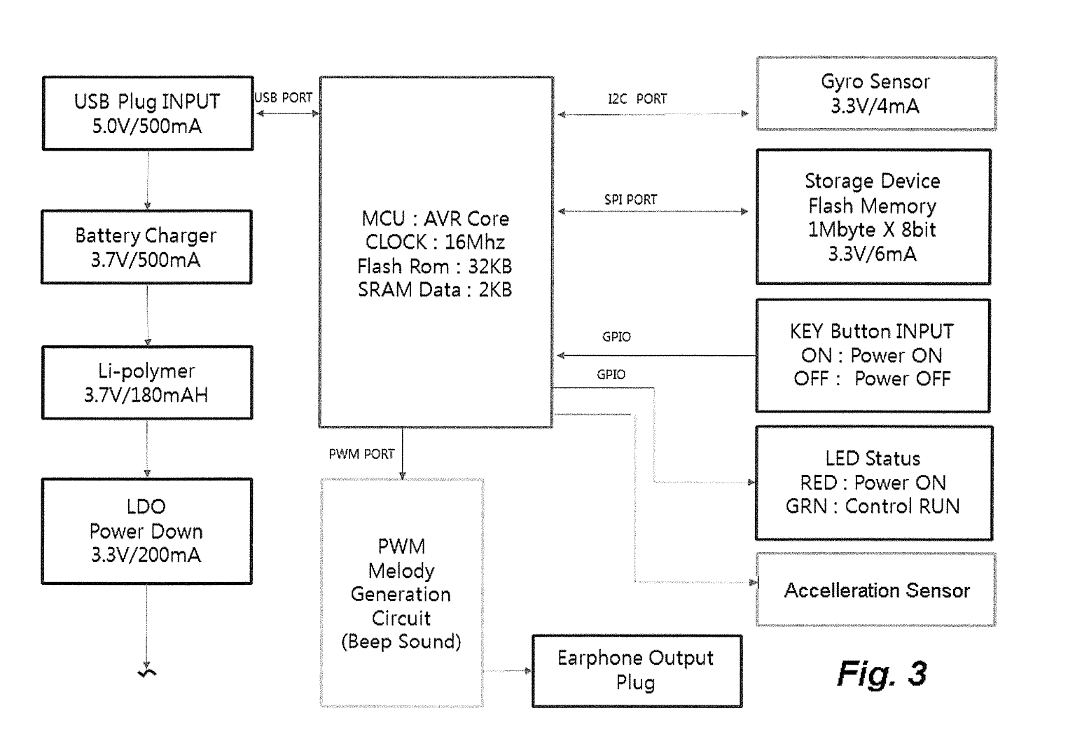

This invention is directed to a neck posture recording and warning device to help users develop safe working postures by warning, recording and/or showing their neck postures as a function of time. FIG. 3 is a block diagram showing important electronic components associated with the device irrespective of packaging which is illustrated in subsequent drawings.

The device is controlled by a microprocessor such as the AVR Core from Amtel Corp. though the invention is not limited in terms of the controller used. In addition to processing functions, the CPU has a clock for determining and recording time-based events, as well as memory for storing results. An additional flash memory storage device is provided for expanded data recording including measured data as a function of time.

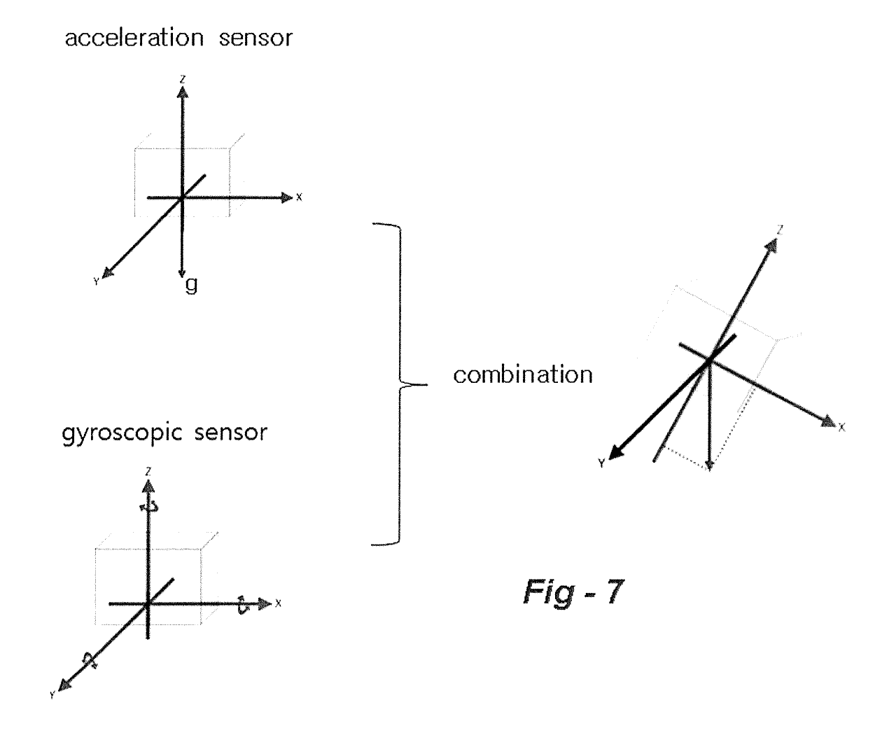

The device further includes an input port such as a USB port that allows for data input and power from a rechargeable battery source. A key button input allows for ON/OFF functions. An LED status indicator shows operational status and may also be used to output alarm status. A melody generation circuit generates alarm sounds, and may be interfaced to a speaker or earphone for private listening. The neck posture warning device further includes a gyro sensor which measures the head tilt angle. In the preferred embodiment, as illustrated in FIG. 7, the neck posture warning device combines an acceleration sensor and a gyroscopic sensor to measure tilt in terms of orientation and rate of change.

In operation, the devices measures head tilt angle and provides an alarm when poor posture is determined. However, the device has several modes of operation. In the most basic mode, the device simply generates an audible and/or visible alarm when head tilt angle exceeds a specific value input by the user (or factory set). In a different mode of operation, the device monitors head tilt over time, and outputs an audible and/or visible alarm if head tilt exceeds a specific value a specific number of times over a specific period also input by the user of factory set. For example, if the user's head tilt exceeds 20 degrees once the device might not issue a warning. However, if head tilt exceeds 20 degrees more than once per minute (for example), an audible and/or visible alarm will be generated.

As a third more than may be used with either of the above, the device may record in memory head tilt over time, regardless of whether the maximum input value has been exceeded. This enables the user to connect a computer or other device such a smartphone to the USB port and prepare charts showing neck tilt behavior over time.

Unique to this invention is the ability to input a particular head tilt value and generate alarms/reports if that value is exceeded. Again, this value may be input by a user (through USB port, for example), or factory set. The specific value will typically be a function of the packaging used for the device, as discussed below. For example, if the unit is attached to eyeglass frames, the critical head tilt angle may be adjusted to account for the reference angle for a particular individual and the type of glasses they are wearing. If packaged as an on-ear or in-device, the device may be rotated or adjusted to account for the angle off-horizontal. In the preferred embodiment, the user may be able to set a head neutral position using pushbuttons provided, such that after such normalization recording will begin. As a further option the device may include an integrated electronic leveling device.

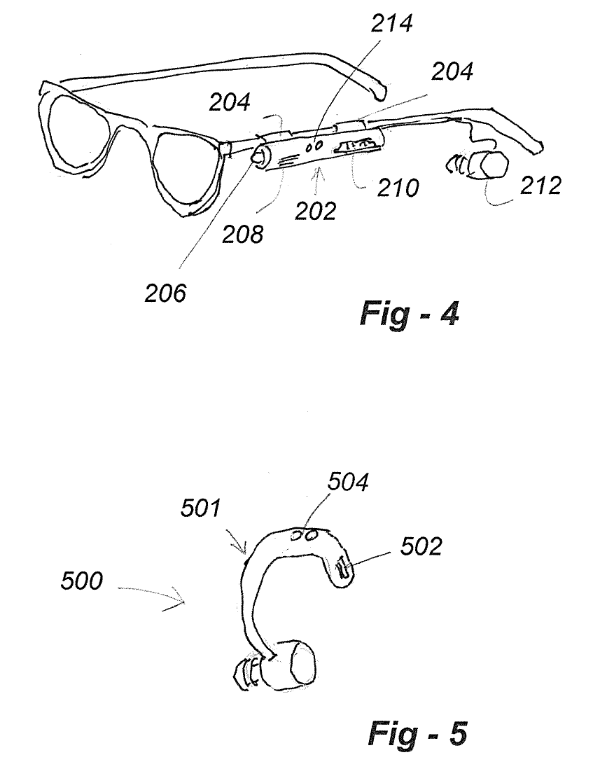

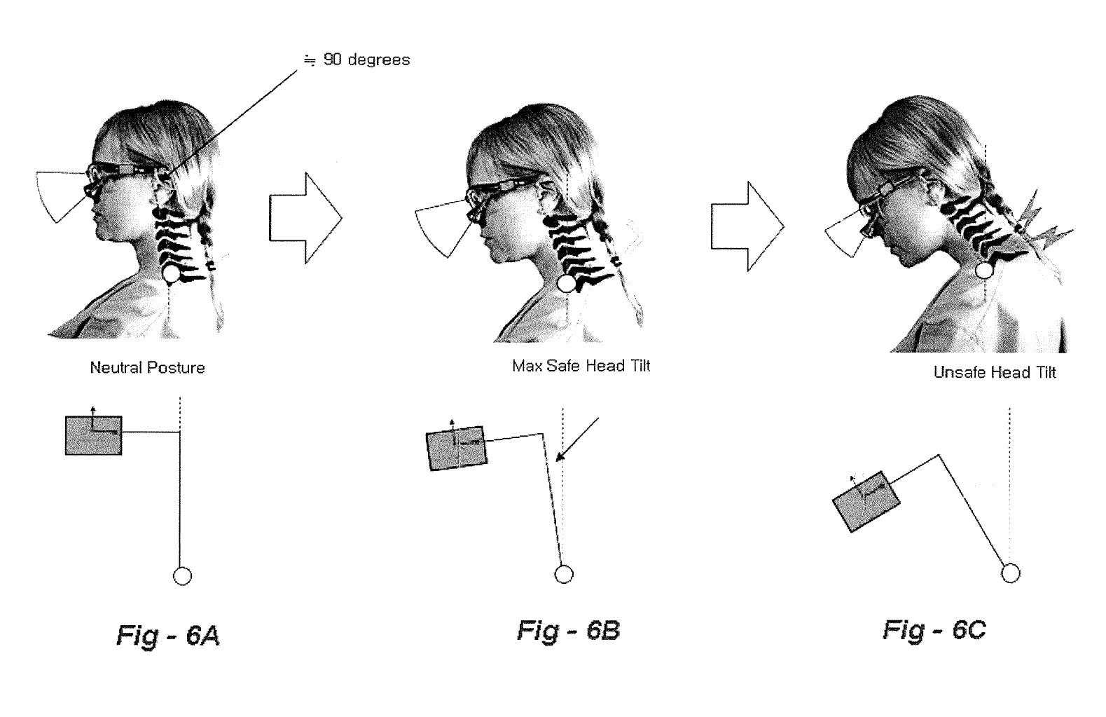

In terms of packaging, FIG. 4 shows the device packaged in a unit 202 that may be attached to a frame temple piece trough one or more clips/fasteners 204. Although the unit is small, current miniaturization allows all of the components to be accommodated. The alarm LED is shown at 204 and the speaker vent at 206. The USB port is shown at 210 and optional earbud, 212. The control buttons are depicted at 214. FIG. 6A is a diagram showing a user wearing an eyeglass-mounted sensor at a neutral posture; FIG. 6B is a diagram showing a user wearing an eyeglass-mounted sensor at a maximum safe head tilt; and FIG. 6C is a diagram showing a user wearing an eyeglass-mounted sensor at an unsafe head tilt. FIG. 5 illustrates an in-ear version of the device at 500 with optional around-ear stabilizer 501. The USB port is shown at 502 and control buttons at 504.

1. A neck posture monitoring device, comprising:
a housing physically coupled to a wearer's head such that the housing moves in a manner corresponding to front-to-back head tilt movements made by the wearer;
a memory disposed in the housing for storing information corresponding to a specific, predetermined forward head tilt angle measured relative to a horizontal reference; and
a sensor disposed in the housing that detects the front-to-back head tilt movements made by the wearer; and
electronic circuitry disposed in the housing operative to (a) compare the front-to-back head tilt movements made by the wearer to the stored specific, predetermined forward head tilt angle, and (b) perform a function when the wearer tilts their head at an angle equal to or greater than the stored particular forward head tilt angle.

2. The device of claim 1, wherein the specific, predetermined forward head tilt angle is between 5 and 35 degrees.

3. The device of claim 1, wherein the specific, predetermined forward head tilt angle is substantially 20 degrees.

4. The device of claim 1, wherein the function performed is to activate an alarm disposed in the housing.

5. The device of claim 4, further including a light source for generating a visible alarm.

6. The device of claim 4, further including a transducer for generating an audible alarm.

7. The device of claim 6, wherein the transducer forms part of an earphone or earbud.

8. The device of claim 1, wherein the housing includes one or more fasteners for attachment to eyeglass frames.

9. The device of claim 1, wherein the housing is an on-ear or in-ear housing.

10. The device of claim 1, further including:
a real-time clock for determining date and time; and
a memory for recording a wearer's head tilt angle as a function of time.

11. The device of claim 10, further including an alarm that is activated when the wearer meets or exceeds the particular forward head tilt angle a specific number of times over a specific period of time.

12. The device of claim 10, further including an alarm that is activated when the wearer meets or exceeds the specific, predetermined forward head tilt angle for a particular duration of time.

13. The device of claim 1, further including an input for receiving information corresponding to a head neutral position.

14. The device of claim 1, further including an electronic level that operates in conjunction with the sensor to determine when a wearer of the device tilt their head at an angle equal to or greater than the specific, predetermined forward head tilt angle.

15. The device of claim 1, wherein the sensor includes a gyroscopic sensor.

16. The device of claim 1, wherein the sensor includes an acceleration sensor.

17. The device of claim 1, wherein the sensor combines a gyroscopic sensor with an acceleration sensor.

18. The device of claim 1, configured as a self-contained battery operated device.
